(19) United States
(12) Patent Application Publication
(10) Pub. No.: US 2010/0331909 A1
(43) Pub. Date: Dec. 30, 2010

(54) ANTI-ARRHYTHMIA IMPLANTABLE MEDICAL DEVICE

(76) Inventors: Karin Järverus, Solna (SE); Andreas Blomqvist, Spanga (SE); Carl Wikefeldt, Solna (SE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/920,906

(22) PCT Filed: Mar. 31, 2008
(86) PCT No.: PCT/SE2008/000238

§ 371 Date: Sep. 3, 2010

(51) Int. Cl. A61N 1/365

(52) U.S. Cl. 607/17

(57) ABSTRACT

An implantable medical device has a detector for detecting an arrhythmia event of a subject's heart and generating an arrhythmia signal based on the detected event. An impedance determining unit determines impedance data representative of blood aggregation level of blood present in a cavity, such as heart chamber, of the subject. An anti-arrhythmia unit of the device is arranged for applying electric anti-arrhythmia treatment to at least a portion of the heart. This unit is conditionally operable based on the arrhythmia signal and the impedance data. The risk blood aggregates and clots obstructing blood vessels following anti-arrhythmia treatment is significant reduces by conditioning the treatment based on the aggregation level representing impedance data.

### BACKGROUND OF THE INVENTION

1. Field of the Invention

The present invention generally relates to an implantable medical device, and in particular to such a device providing anti-arrhythmia treatment and to an anti-arrhythmia treatment method.

2. Description of the Prior Art

The perfusion of blood through living tissue is a central mechanism for maintaining life. The formation of blood clots is a major concern within treatment of cardiovascular diseases, particularly in the arrhythmia field. Patients suffering from different types of arrhythmia often have an increased risk of formation of blood clots. Such blood clotting is of course a large problem to these patients as it negatively effects the necessary blood perfusion through various tissues and organs.

Traditional anti-arrhythmia treatment devices and protocols may actually increase the dangerous risks associated with blood clotting in connection with arrhythmia patients. Indeed their applied anti-arrhythmia treatment may propel formed clots out into arteries, where they may cause a series of severe conditions, including ischemia.

### SUMMARY OF THE INVENTION

There is, thus, a need for an implantable medical device and treatment method that provides an anti-arrhythmia treatment that is adapted to the problems associated with blood aggregation and clotting. The present invention overcomes the drawbacks of the prior art arrangements.

It is a general object of the present invention to provide an efficient implantable medical device capable of providing a conditional anti-arrhythmia treatment.

It is another object of the invention to provide an anti-arrhythmia treatment that is conditional on the blood aggregation and/or clotting status.

Briefly, the present invention involves an implantable medical device having an arrhythmia detector for detecting an arrhythmia event of a subject's heart. This detector performs the detection based on electrical signals sensed from the heart. If an arrhythmia event is detected, the detector generates an arrhythmia signal indicative of that event. An impedance determining unit is arranged in the device for determining impedance data, preferably in response to the arrhythmia signal. This impedance data is representative of the erythrocyte aggregation level in blood present in a body cavity in the subject body. The cavity is preferably in connection with or forms part of the cardiovascular system of the subject and can advantageously be a heart chamber.

The implantable medical device also has an anti-arrhythmia unit arranged for applying, using implantable electrical leads connectable to the device, electric anti-arrhythmia treatment, such as a defibrillation shock, cardioversion or anti-arrhythmia pacing, to at least a portion of the heart. This anti-arrhythmia unit is, according to the invention, conditionally operable based on the arrhythmia signal and the impedance data. This conditional operation means that the current blood aggregation status of the patient is taken into consideration before applying any anti-arrhythmia treatment. As a consequence, an alternative treatment, a postponement of the anti-arrhythmia treatment or even a cancellation thereof can be selected by the anti-arrhythmia unit if the impedance data indicates a high blood aggregation level. At this high level there is a significant risk of the presence of blood aggregates and clots that can obstruct blood vessels, including coronary vessels, if the anti-arrhythmia treatment would be applied immediately.

In a preferred embodiment, electric anti-arrhythmia treatment is applied to the heart by the implantable medical device if the impedance data indicates low risk for the presence of dangerous, potentially obstructing blood aggregates or clots and/or the arrhythmia is regarded as fatal, such as ventricular fibrillation. However, if the arrhythmia is potentially non-fatal and the impedance data indicates high aggregation/clotting risk, the treatment can be postponed or cancelled, allowing the subject to take anti-clotting actions, such as anticoagulants or anti-thrombotic agents, before applying any anti-arrhythmia treatment.

The present invention also involves a method for treating an arrhythmia event in a subject, where the treatment is conditional based on erythrocyte aggregation status as represented by determined impedance data.

The invention offers the following advantages:
Reduces the risk of blood vessel obstructions in connection with anti-arrhythmia treatment; and
Can be implemented using existing sensor technology present in implantable medical devices.

Other advantages offered by the present invention will be appreciated upon reading of the below description of the embodiments of the invention.

### BRIEF DESCRIPTION OF THE DRAWINGS

The invention together with further objects and advantages thereof, may best be understood by making reference to the following description taken together with the accompanying drawings.

FIG. 1 is a schematic overview of a subject having an implantable medical device according to the present invention.

FIG. 2 is a schematic block diagram of an implantable medical device according to an embodiment of the present invention.

FIG. 3 is a schematic block diagram of an implantable medical device according to another embodiment of the present invention.

FIG. 4 is a schematic block diagram of an impedance measuring unit according to an embodiment of the present invention.

FIG. 5 is a flow diagram illustrating a method of treating an arrhythmia state according to the present invention.

FIG. 6 is a flow diagram illustrating an additional step of the treatment method in FIG. 5.

FIG. 7 is a flow diagram illustrating the impedance determining step of FIG. 5 in more detail according to a particular embodiment of the present invention.

FIG. 8 is a flow diagram illustrating additional steps of the treatment method in FIG. 5.

FIG. 9 is a flow diagram illustrating the treatment decision step of FIG. 5 in more detail according to a particular embodiment of the present invention.

### DESCRIPTION OF THE PREFERRED EMBODIMENTS

Throughout the drawings, the same reference characters will be used for corresponding or similar elements.

The present invention generally relates to implantable medical devices and methods for providing a conditional treatment of arrhythmia events of an animal subject, preferably a mammalian subject and more preferably a human subject.

Cardiac arrhythmia is characterized by abnormal electrical activity in the heart. Such cardiac arrhythmias are associated with a particular adverse event in connection with the blood status of the cardiovascular system. Thus, an arrhythmia, whether resulting in too fast, too slow or too weak heart beats, can negatively affect the blood circulation in the cardiovascular system and thereby promote blood aggregation and clotting.

In a simplistic disclosure, the impaired blood circulation, due to the arrhythmia condition or state, causes erythrocytes to aggregate forming erythrocyte aggregates or clusters as described in Muralidharan, “Kinetics of erythrocyte aggregation in myocardial infarction”, Proceedings of the Annual International Conference of IEEE Engineering in Date, 9-12 Nov. 1989, pages 873-874, volume 3. Such blood cell aggregates can be in the form of stacks, called “rouleaux”, of red blood cells. The erythrocyte aggregation causes an increase in the blood viscosity. The aggregation and the blood viscosity increase subsequently lead to formation of blood clots and possibly later thrombosis. Arrhythmia also often leads to impaired or reduced blood flow velocity. This reduced flow further triggers and promotes erythrocyte aggregation and viscosity increases.

If an anti-arrhythmia treatment is initiated following such a high aggregation condition, the formed blood aggregates and clots may be propelled out into arteries and blood vessels once the arrhythmia has stopped and normal blood flow velocity is resumed. The blood clots can therefore become stuck into and obstruct arteries and other blood vessels, leading to ischemia in the nearby tissue. The erythrocyte aggregation and clot formation can particularly occur in a heart chamber, such as in the left atrium or ventricle. In such a case, a propelled blood clot may indeed partly obstruct the blood supply to a part of the heart, causing ischemia and potentially myocardial infarction.

The present invention provides a solution to this increased risk of anti-arrhythmia treatment due to the formation of blood aggregates and clots by providing an anti-arrhythmia treatment that is being conditional based on the erythrocyte aggregation status or level of the subject. This means that another anti-arrhythmia treatment strategy can be employed in the case of high aggregation status as compared to low aggregation status to thereby prevent or at least reduce the risk of subsequent blood supply obstruction due to the anti-arrhythmia treatment.

Impaired blood circulation and erythrocyte aggregation can occur both in connection with too fast, too slow and irregularly beating hearts. As a consequence, the present invention can be applied in connection with both tachyarrhythmias (generally having a heart rate faster than 100 beats per minute in adults) and bradyarrhythmias (generally having a heart rate slower than 60 beats per minute in adults). Other forms of arrhythmias, such as atrial or ventricular fibrillation may also cause erythrocyte aggregation and increased risk for blood clot formation and blood vessel obstruction.

The present invention utilizes impedance measurements collected by an implantable medical device as indicative or representative of the erythrocyte aggregation level of the subject's blood. The erythrocyte aggregation causes, as was mentioned above, an increase in blood viscosity. Such a viscosity increase is closely related to a corresponding impedance increase as described in Zhao, “Electrical impedance of human blood”, PhD thesis at Karolinska Institutet, Sweden, 1993. Furthermore, blood cells, including erythrocytes, tend to align in a blood vessel at higher velocity as compared to the erratic behavior during lower velocities associated with arrhythmia conditions. This non-alignment of the blood cells correlates with an impedance increase as described in U.S. Pat. No. 6,673,622 and U.S. Pat. No. 4,947,678. In summary, blood aggregation causes a viscosity raise of the blood, which is detectable through impedance measurements. Furthermore, arrhythmia often causes an impaired (reduced) blood flow velocity, which is also detectable through impedance measurements. Thus, impedance measurements can be effectively used to determine or at least estimate erythrocyte aggregation status of the blood in a subject.

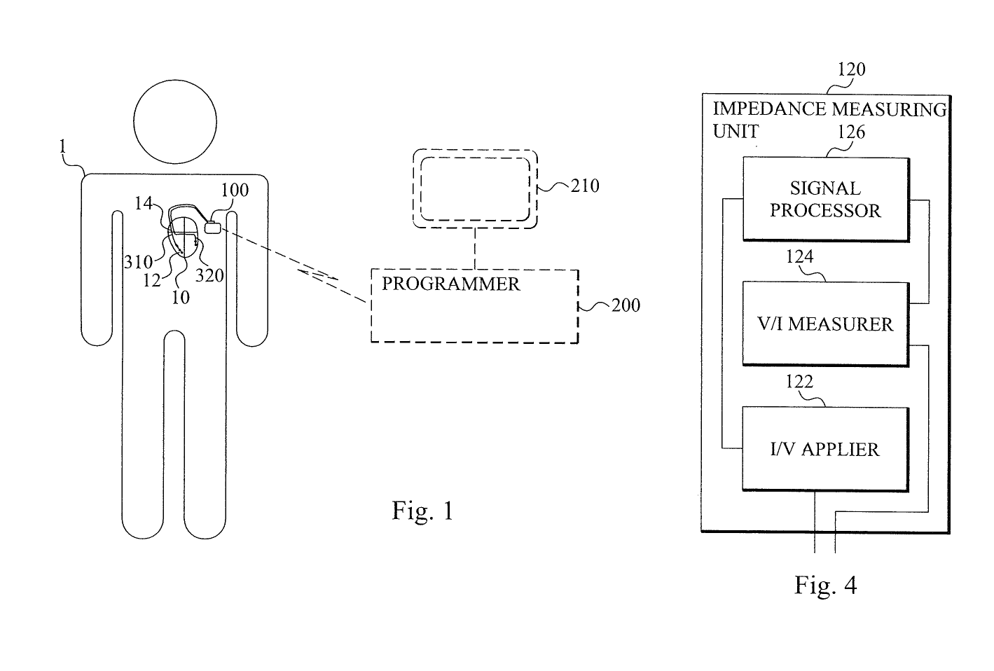

FIG. 1 is a schematic overview of a patient 1 having an implantable medical device, IMD, 100 according to the present invention. In the figure, the IMD 100 is illustrated as a device that monitors and/or provides therapy to the heart 10 of the patient 1, such as a pacemaker, cardiac defibrillator or cardioverter. The IMD 100 is, in operation, connected to one or more, two in the figure, cardiac leads 310, 320 inserted into different heart chambers, the right and left ventricles 12 in the figure. The present invention is though not limited to ventricular leads 310, 320 but can also be used in connection with leads positioned in the right or left atrium 14 of the heart 10. Actually, also non-intracardiac leads, including epicardiac leads can also be used.

FIG. 1 also illustrates an external programmer or clinician's workstation 200 that can communicate with the IMD 100. As is well known in the art, such a programmer 200 can be employed for transmitting IMD programming commands causing a reprogramming of different operation parameters and modes of the IMD 100. Furthermore, the IMD 100 can upload diagnostic data descriptive of different medical parameters or device operation parameters collected by the IMD 100. Such uploaded data may optionally be further processed in the programmer 200 before display to a clinician on a connected display screen 210. In the light of the present invention, such diagnostic data can include erythrocyte aggregation data descriptive of the blood aggregation status in the patient 1 measured by the IMD 100 and/or other diagnostic data relating to arrhythmia detection, classification and/or treatment.

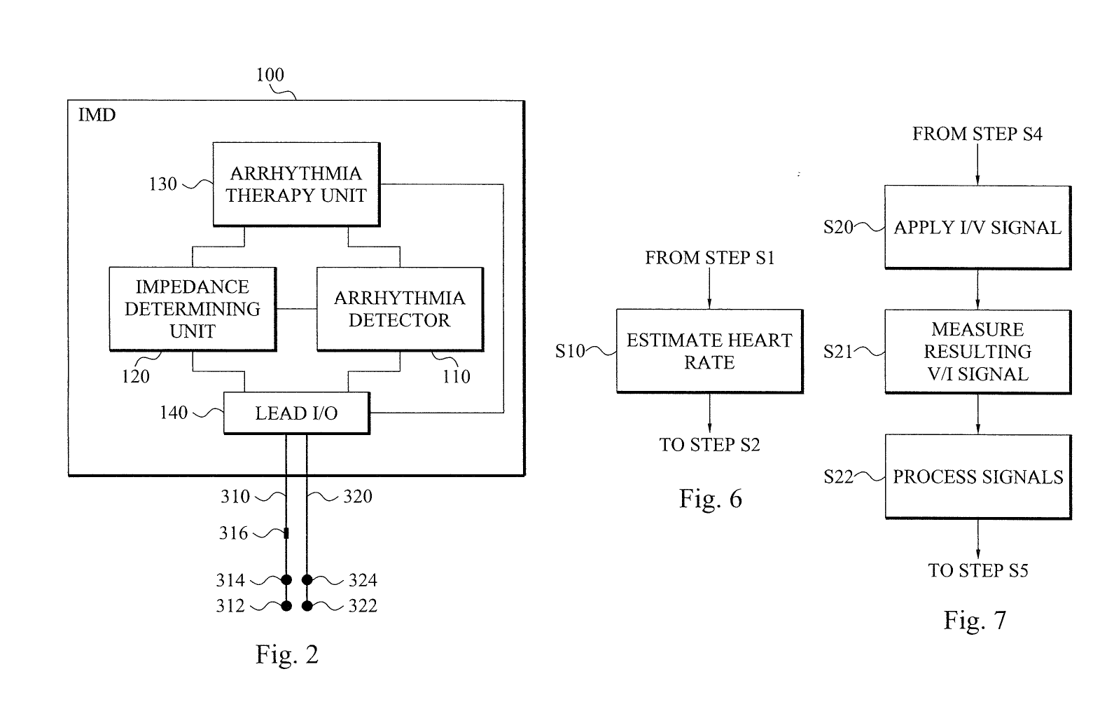

FIG. 2 is a schematic block diagram of an IMD 100 according to the present invention. The IMD 100 comprises an arrhythmia detector 110 arranged for detecting the presence of an arrhythmia state or condition in a heart of a subject. This arrhythmia detector 110 is preferably connected to a lead input 140 of the IMD 100. The lead input 140 is in turn connectable to one or more cardiac leads 310, 320, preferably intracardiac leads 310, 320. These leads 310, 320 each have one or more electrodes 312, 314, 316; 322, 324 for sensing and measuring different electrical parameters in the subject's heart and/or applying electrical pulses or shocks to the heart. In this context, different forms of lead electrodes well-known in the art can be used, including lead tip electrodes 312, 322, lead ring electrodes 314, 324 and lead coil electrodes 316. The electrodes 312, 314, 316; 322, 324 preferably measure or sense intracardiac electrical signals, which are forwarded through the leads 310, 320 and lead input 140 to the arrhythmia detector 110 for processing. The detector 110 then uses these intracardiac electric signals for detecting the presence of an arrhythmia. These electric signals preferably represent electrocardiogram (ECG) signals and preferably intracardiac electrocardiogram (IEGM) signals.

For example, the collected and processed intracardiac electrocardiogram signals could represent or be representative of the ventricular beating rate. In such a case, the detector 110 can signal a tentative arrhythmia if the sensed ventricular rate exceeds or falls below a pre-defined threshold rate value or range. The detection can preferably be at least partly time-based if, for example, the detector 110 collects ventricular rate data over a time interval and determines an average value. This average value can then be compared to the threshold value/range. In an alternative approach, the detector 110 compares multiple collected ventricular rate values with the threshold and signals a tentative arrhythmia if at least a minimum selected portion of the rate values exceed or fall below the threshold value/range.

The arrhythmia detector 110 preferably generates an arrhythmia signal if an arrhythmia state is detected, for instance, based on the threshold-rate comparison. This arrhythmia signal is therefore indicative of the presence of an arrhythmia event of the subject's heart.

The IMD 100 also comprises an impedance determining unit 120 preferably connected to the arrhythmia detector 110 and the lead input 140. This determining unit 120 is arranged for determining, based on electrical signals, impedance data representative of an erythrocyte aggregation or blood clotting level of blood present in a blood-containing cavity in the subject. The cavity is preferably connected to the cardiovascular system of the subject and can advantageously be a heart chamber. Thus, preferred blood-containing cavities, in which impedance measurements are conducted, include the left atrium, the left ventricle, the right atrium and the right ventricle. The present invention is though not limited to heart cavities but can be used in connection with other blood-containing cavities in the subject body, including the IMD pocket.

The applying and sensing electrodes 312, 314, 316; 322, 324 employed for performing the impedance measurements are preferably arranged relative the cavity to provide impedance data of the (flowing) blood present in the cavity. This means that the impedance data closely follows (through the above-mentioned dependencies between aggregation, blood velocity and impedance) the current aggregation status of the blood flowing through the cavity.

In a preferred embodiment, the impedance determining unit 120 is responsive to the arrhythmia signal from the arrhythmia detector 110. Thus, the determining unit 120 preferably collects and processes electrical signals in response to reception of such an arrhythmia signal to generate the impedance data. In an alternative approach, though requiring more battery and processing power, the impedance determining unit 120 can be configured for performing periodic or intermittent impedance measurements instead of or as a complement to the arrhythmia triggered impedance determination. Such timed impedance measurements can be scheduled to occur from a few times per hour or even more often down to one or a few times per day.

The determining unit 120 preferably measures the impedance during multiple time periods of different heart beats. For example, the determining unit 120 could determine impedance values for several successive heart beats or for several timely separated heart beats occurring in a defined time interval. These different impedance values over different heart beats can then be collectively processed by the determining unit 120 to form average impedance data. It is also possible to use a floating average value calculation so that the determined impedance data is an average of the impedance values collected during the last N measured heart intervals, where N is a predefined integer equal to or larger than two, preferably 3-20. Thus, usage of such a (floating) average reduces the variability in impedance signal that can occur during arrhythmia situations.

The determined impedance signal can be a complex impedance signal, i.e. comprising both a real and imaginary part. These respective parts can then be separately or collectively processed by the impedance determining unit 120. For example, the real and imaginary parts can be employed for calculating a phase angle between the sensed current and applied voltage. Alternatively, the real and imaginary parts are employed for calculating the amplitude of the impedance. As a consequence, the impedance data determined by the impedance determining unit 120 and employed by the present invention can be phase angle data, amplitude data or both phase angle and amplitude data.

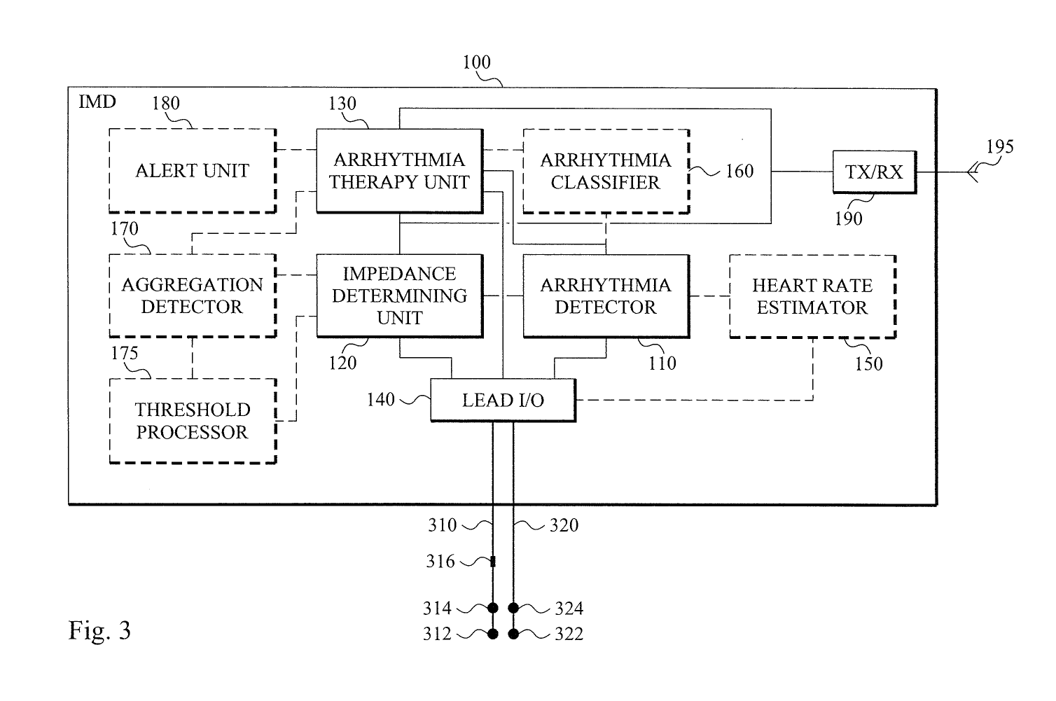

FIG. 4 is a schematic block diagram illustrating a possible implementation of the impedance determining unit 120 of FIG. 2 (or FIG. 3). The determining unit 120 comprises a signal applier 122 for applying, over two electrodes of the lead(s) connectable to lead input of the IMD, an electrical signal to the blood present in the measuring cavity. This signal applier 122 is preferably responsive to the arrhythmia signal from the arrhythmia detector. As a consequence, the applier 122 applies the signal to the cavity portion lying between the selected lead electrodes based on reception of this arrhythmia signal.

The signal applier 122 can be arranged for generating and applying a predefined current or voltage signal. As is known in the art, such an applied current or voltage signal, preferably current signal can be a stepwise or gradually changing (current) signal.

A signal measurer 124 is implemented in the determining unit 120 for measuring, using at least two electrodes of the lead(s) connectable to the lead input of the IMD, a resulting electrical signal over at least a portion of the heart. This measurer 124 preferably measures a resulting voltage signal, if the signal applier 122 applied a current signal or pulse, and measures a resulting current signal, if the applier 122 applied a voltage signal.

The impedance determining unit 120 also comprises a signal processor 126 for determining the impedance data based on the electrical signal applied by the signal applier 122 and the resulting electrical signal measured by the signal measurer 124. The signal processor 126 employs well known signal processing techniques for determining the impedance data based on the raw input electrical signals. Briefly, the input measured AC voltage is optionally pre-amplified and an integrated by calculating the voltage area of the signal per pulse. The applied AC current signal is also integrated by calculating the current area of the signal per pulse. The integrated absolute impedance can then be calculated in block as the quotient between the voltage area and the current area. This raw impedance signal may be further processed in a filter chain. The filter output is A/D converted to form the desired output impedance signal.

The units 122 to 126 of the impedance determining unit 120 may be provided as hardware, software or a combination of hardware and software. A distributed implementation is also possible where at least one of the units 122 to 126 is implemented elsewhere in the IMD.

The present invention can be used in connection with both bipolar, tripolar and quadropolar impedance vectors that are reflective of the erythrocyte aggregation level of the blood. As is well known in the art, in bipolar impedance measurements the same two electrodes are used for both current/voltage application and voltage/current sensing. In tripolar one electrode is common for the signal application and sensing while the two other electrodes are used solely for signal application or signal sensing. Finally quadropolar measurements use dedicated signal application electrodes and dedicated signal sensing electrodes. In particular bipolar and tripolar impedance vectors are preferred.

For instance, if the relevant blood-containing cavity is the right atrium a bipolar impedance vector between tip and ring electrodes of a right atrial lead can be used. Corresponding examples of a tripolar impedance vector could be by applying the current/voltages between the right atrial ring (or tip) electrode and the case (IMD body electrode) and sense the resulting voltage/current between the right atrial tip (or ring) electrode. The usage of tip/ring/case electrodes can also be applied in connection with a right ventricular lead, left atrial lead and left ventricular lead.

The implantable medical device 100 of FIG. 2 also comprises an anti-arrhythmia therapy unit 130. This therapy unit 130 is arranged for applying, using at least one lead 310, 320 connectable to the lead input/output (I/O) unit 140, electric anti-arrhythmia treatment to at least a portion of the subject's heart. There are several different forms of electrical anti-arrhythmia treatments applicable by IMD, which are well-known in the art. For instance, pacemakers can apply anti-arrhythmia pacing therapy to the heart to thereby treat the arrhythmia event.

Implantable cardioverter-defibrillators can correspondingly apply electrical cardioversion or defibrillation shocks to the heart. Any such well-known IMD-based electrical anti-arrhythmia treatment disclosed in the art can be employed by the arrhythmia therapy unit 130.

In clear contrast to traditional IMDs 100 with anti-arrhythmia therapy units 130, the therapy unit 130 of the present invention is conditionally operable based on the arrhythmia signal from the arrhythmia detector 110 and the impedance data from the impedance determining unit 120. Traditionally, IMDs 100 apply anti-arrhythmia treatment once an arrhythmia detector 110 detects an arrhythmia event. However, the present invention utilizes a conditional therapy unit 130 to determine whether an anti-arrhythmia treatment should be initiated or not and preferably also what type of treatment type to performed based on the impedance data. This means that the anti-arrhythmia treatment of the invention is conditional upon the current erythrocyte aggregation and blood clotting level of the subject's blood. Such aggregation-based and -conditional therapy unit 130 can therefore, if the impedance data represents a high aggregation level and thereby a large risk for blood clots, elect to not initiate any anti-arrhythmia treatment or perform an alternative anti-arrhythmia treatment, such as postpone the initiation of the application of the electrical anti-arrhythmia treatment.

Thus, in an embodiment of the present invention, the anti-arrhythmia unit 130 has access to at least two anti-arrhythmia treatment schemes that can be effected based on the arrhythmia signal from the arrhythmia detector 110 and the impedance data from the impedance determining unit 120. In such a case, the first treatment scheme is performed by the therapy unit 130 if the impedance data indicates a low aggregation level of the blood and thereby a low risk for thrombosis. The treatment scheme can then involve a traditional anti-arrhythmia treatment, such as applying, through the lead output 140 and the leads 310, 320 a defibrillation or cardioversion shock or anti-arrhythmia pacing. However, if the impedance data indicates a comparatively higher aggregation level and thereby a significant risk for thrombosis, the anti-arrhythmia unit 130 instead selects the second treatment scheme. An example of such a scheme could be to postpone the application of electrical anti-arrhythmia signals to the heart at least for a defined period of time, allowing the patient to take anti-thrombotic medicines and/or visit his/her physician before any therapy application. Another example is to provide another pacing sequence that reduces the risk of obstructing any blood vessels with formed blood aggregates or clots.

The units 110 to 140 of the IMD 100 may be provided as hardware, software or a combination of hardware and software. In the figure only those IMD units 110 to 140 directly involved in the present invention have been indicated. It is anticipated by the present invention that the IMD 100 also comprises other units and functionalities directed to its operation but not directly involved in the invention.

FIG. 3 is a schematic block diagram of another embodiment of an IMD 100 according to the present invention. This IMD 100 preferably comprises a heart rate estimator 150 arranged connected to the lead input 140. This estimator 150 estimates a heart rate of the heart based on (intrinsic) electrical signals from the heart and sensed by at least one of the leads 310, 320. The estimator 150 can advantageously estimate the heart rate from a measured electrocardiogram signal obtained from the sensed electrical signals. In such a case, the arrhythmia detector 110 is arranged for detecting the presence of an arrhythmia condition based on a comparison of the estimated heart rate and at least one threshold rate or interval as previously described.

In a preferred embodiment, the IMD 100 also comprises an arrhythmia classifier 160 connected to the arrhythmia detector 110 and arranged for processing the arrhythmia signal from the detector 110. This arrhythmia classifier 160 uses the arrhythmia signal for classifying the detected arrhythmia condition into at least two different arrhythmia classes: potentially fatal arrhythmia condition and potentially non-fatal arrhythmia condition.

According to the present invention, a fatal arrhythmia condition is a life threatening condition that should be treated immediately following detection. An example of such a condition is ventricular fibrillation. If left untreated, ventricular fibrillation (VF) can lead to death within minutes. When a heart goes into VF, effective pumping of the blood stops. VF is generally considered a form of cardiac arrest and an individual suffering from it will not survive unless anti-fibrillation treatment, such as defibrillation, is provided immediately.

Non-fatal arrhythmia includes different forms of arrhythmias that are not immediate life-threatening. As a consequence, anti-arrhythmia treatment must not necessarily be initiated immediately following detection of such a non-fatal arrhythmia condition without significant risk to the patient's health. Examples of arrhythmia conditions that could be regarded as non-fatal include atrial fibrillation, bradycardia, and tachycardia.

The classifier 160 processes the arrhythmia signal from the detector 110, where this signal contains characteristic features of the electrical activity of the heart. For instance, the classifier 160 can classify the arrhythmia as ventricular fibrillation (fatal arrhythmia condition) due to a turbulent, disorganized electrical activity of the heart in such a way that recorded electrocardiographic deflections continuously change in shape, magnitude and direction. Thus, in particular if multiple electrodes 312, 314, 316; 322, 324 present in one or more leads 310, 320 are connected to the IMD 100, a more complete picture of the electrical activity at different portions of the heart can be sensed to thereby facilitate in the arrhythmia classification. For more information of arrhythmia classification, reference is made to U.S. Pat. No. 5,042,497, United States Patent Application Publication No. 2005/0154421 and International Patent Application No. PCT/SE2007/000581.

In either case, the arrhythmia classifier 160 generates classification data representative of the estimated severity of the detected arrhythmia condition. This data can a simple discrimination between potentially fatal or non-fatal arrhythmia as mentioned above. In other embodiments, a more detailed arrhythmia classification that, for instance, discriminates between atrial and ventricular fibrillation, atrial and ventricular bradycardia and atrial and ventricular tachycardia can be used in the classification data.

The arrhythmia therapy unit 130 is then connected to the classifier 160 and is conditionally operable based on the arrhythmia signal and the impedance data as discussed above but preferably also based on the classification data. In such a case, if the classification data indicates a potentially fatal arrhythmia state, the arrhythmia therapy unit 130 is operable for applying the electric anti-arrhythmia treatment to at least a portion of the heart using at least one connected lead 310, 320. This therapy application is then conducted regardless of the current aggregation status as the arrhythmia is potentially lethal to the patient and should be combated as soon as possible. However, if the classification data indicates a potentially non-fatal arrhythmia condition, the anti-arrhythmia unit 130 is operable for conditionally applying the electric anti-arrhythmia treatment to the heart based on the arrhythmia signal and the impedance data. This means that for a not immediately life-threatening state, the therapy unit 130 first investigates the aggregation status of the blood before initiating or selecting any anti-arrhythmia treatment. In this case, it could be better to postpone any anti-arrhythmia treatment to allow the patient to first take an anticoagulant or anti-thrombotic medicine before applying the electrical treatment to the heart.

An aggregation detector 170 is preferably implemented in the IMD 100 connected to the impedance determining unit 120. This detector 170 is arranged for comparing the impedance data from the determining unit 120 with a stored impedance threshold or template. The threshold or template preferably represents impedance data indicative of a normal (low) erythrocyte level of the blood. As a consequence, an aggregation risk is present if the determined impedance data differs significantly from the impedance threshold or template. If an impedance amplitude value is employed as impedance data, there is a high aggregation risk if the impedance value significantly exceeds the threshold value in this example. It is of course possible to instead utilize an impedance threshold or template representative of a significant risk for erythrocyte aggregation. In such a case, a normal/healthy aggregation level is present if there is a significant difference between the impedance data and the threshold/template.

If the aggregation detector 170 detects a currently high erythrocyte aggregation level 170 it signals the arrhythmia therapy unit 130 that then conditions the anti-arrhythmia treatment based on the signal and the impedance-threshold/template comparison.

As is well known in the art, there are several (external) factors that can effect impedance measurements, including patient posture, patient activity level, etc. In such a case, different threshold/templates can be used for different postures and/or activity levels. The IMD 100 then preferably comprises or is connected to a posture sensor and/or and activity sensor. In such a case, the aggregation detector 170 receives both the impedance data from the impedance determining unit 120 and posture/activity data from the posture/activity sensor. The posture/activity data is employed for selecting the correct impedance threshold/template to compare with the impedance data based on the patient posture/activity level during the impedance measurements.

In a more elaborated embodiment, the IMD 100 also has a threshold or template processor 175 connected to the impedance determining unit 120. The threshold processor 175 receives impedance data collected by the determining unit 120 during normal (healthy) aggregation status and heart status. This received impedance data is processed by the threshold processor 175 for the purpose of determining one or more impedance templates or thresholds that are stored in a connected memory (not illustrated). The threshold(s) can then be used by the aggregation detector 170 together with impedance data from the determining unit 120 to determine the current erythrocyte aggregation status of the patient's blood.

The template or threshold is preferably determined as an average impedance template/threshold through averaging impedance data collected over multiple heart beats. A template will then be a waveform over the average impedance changes during a heart beat and a threshold will be an average impedance parameter from multiple heart beats.

It is anticipated by the present invention that the same impedance vector is preferably employed for generating the impedance template/threshold as for determining the impedance data.

The threshold processor 175 preferably regularly updates the impedance template or threshold over time to reflect impedance changes caused by other factors than arrhythmia and erythrocyte aggregation, such as changes to the local tissue environment or changes to the lead 310, 320 or lead electrodes 312, 314, 316; 322, 324 employed for collecting the raw electric signals. This means that the impedance template/threshold could for example be updated daily, weekly, monthly or even more seldom.

In a preferred implementation, the threshold processor 175 determines a set of multiple different standard impedance templates or thresholds. The respective impedance templates/thresholds can then be adapted to different patient states or conditions, which affect the impedance signals, including body position and body activity as mentioned above. In the latter case, the IMD 100 preferably has a heart rate estimator 150 connected to the lead input 140. The heart rate estimated by the estimator 150 can be used as a representation of a current body activity. It is anticipated by the present invention that other types of activity sensors, including accelerometers and respiratory rate determining units (could use thoracic impedance data), could instead be used.

In either case, impedance data is collected by the impedance measuring unit 120 as the heart rate estimates 150 determines the current heart rate. The threshold processor 175 then uses the collected impedance data and the heart rate for generating different impedance templates/thresholds, where each such template/threshold is representative of the normal impedance at a given heart rate interval. The template memory will then contain a set of different such impedance templates/thresholds with different associated heart rate intervals.

Correspondingly, the IMD 100 can also or instead comprise a posture sensor. In similarity to the heart rate estimator 150, the posture sensor can determine different current body postures, such as standing, lying (on the back, on the stomach (in prone position), on the left side or on the right side) or sitting, in connection with collecting impedance data. In such a case, different impedance templates/thresholds can be generated by the threshold processor 175 for different body postures and stored in the memory.

The two embodiments described above can of course be combined. In such a case, an impedance template/threshold can be associated with both a body posture and a heart rate interval.

An alert unit 180 is preferably provided in the IMD 100 connected to the arrhythmia therapy unit 130. This alert unit 180 is arranged for providing an alert signal if the anti-arrhythmia unit 130 selects an alternative anti-arrhythmia treatment scheme, such as postponing the application of the electric anti-arrhythmia treatment, based on the impedance data. In a first embodiment, the alert signal is forwarded to a transmitter or transceiver 190 having connected antenna means 195 for wirelessly transmitting data to an external (non-implanted) unit. The transmitter 190 sends the alert signal to the external unit, such as the programmer of FIG. 1, a home monitoring system and/or a portable unit, including mobile telephone, personal digital assistant, laptop or dedicated handheld communications terminal, having capability to wirelessly receive data from the IMD 100. In such a case, the external unit preferably runs an alarm, such as display a message, provides an audio alarm, etc. that notifies the patient or his/her physician that an arrhythmia event has been detected and the current erythrocyte aggregation level is dangerously high. This then allows the patient to take actions to combat the aggregation problem, such as taking anticoagulant or anti-thrombotic medicine, as mentioned above.

In addition or alternatively, the alert unit 180 could itself generate a tactile and/or audio alert signal. In the former case, the IMD case or body, or at least a portion thereof, vibrates, which is sensed by the patient. In the latter case, the patient will hear the generated audio signal. The patient has furthermore previously been informed that such a tactile and/or audio alert is sounded/run in the case of a detected arrhythmia condition in connection with high aggregation level.

The usage of an alert unit 180 is particularly advantageous in connection with a conditional anti-arrhythmia treatment in the form of postponing a planned application of an electric anti-arrhythmia signal to the heart. In such a case, the alert urges to the patient to take necessary anti-aggregation actions before the electric anti-arrhythmia signal is applied by the therapy unit 130. This can be realized by starting a clock at the therapy unit 130 based on the arrhythmia signal (presence of a non-fatal arrhythmia) and the impedance data (high erythrocyte aggregation level). Once the clock has counted down, the postponed anti-arrhythmia treatment can be applied directly. Alternatively, the therapy unit 130 performs a new conditional check based on newly acquired arrhythmia signal and impedance data. If the signal indicates the presence of an arrhythmia but the impedance data indicates a reduction of the aggregation level, the anti-arrhythmia treatment can be safely applied. If the impedance data, however, still indicates dangerous aggregation level, the therapy unit 130 can reset the clock and wait a new pre-defined time period to allow any anti-aggregation actions to take effect. This clock resetting can possibly be performed a maximum number of times and then electric anti-arrhythmia treatment, preferably aggregation-adjusted electric anti-arrhythmia treatment, is applied even though the impedance data indicates that the aggregation level has not yet resumed normal values.

The units 110 to 190 of the IMD 100 may be provided as hardware, software or a combination of hardware and software.

The impedance data representative of erythrocyte aggregation level collected according to the present invention can be of diagnostic value in addition to be used in the conditioning of the anti-arrhythmia treatment. Thus, information of changes in the aggregation status of the blood in a patient over time provides highly diagnostic values for the physician. The data can for instance be used in prescribing medicines, treatment planning, etc. for the patient. As a consequence, impedance data can be generated by the impedance determining unit 120 also at occasions with no detected arrhythmia. The generated data is then stored in a memory (not illustrated) of the IMD. Once the patient visits his/her physician, such impedance data can be transmitted by the transmitter 190 to the physician's programmer or workstation and form part of the medical history of the patient.

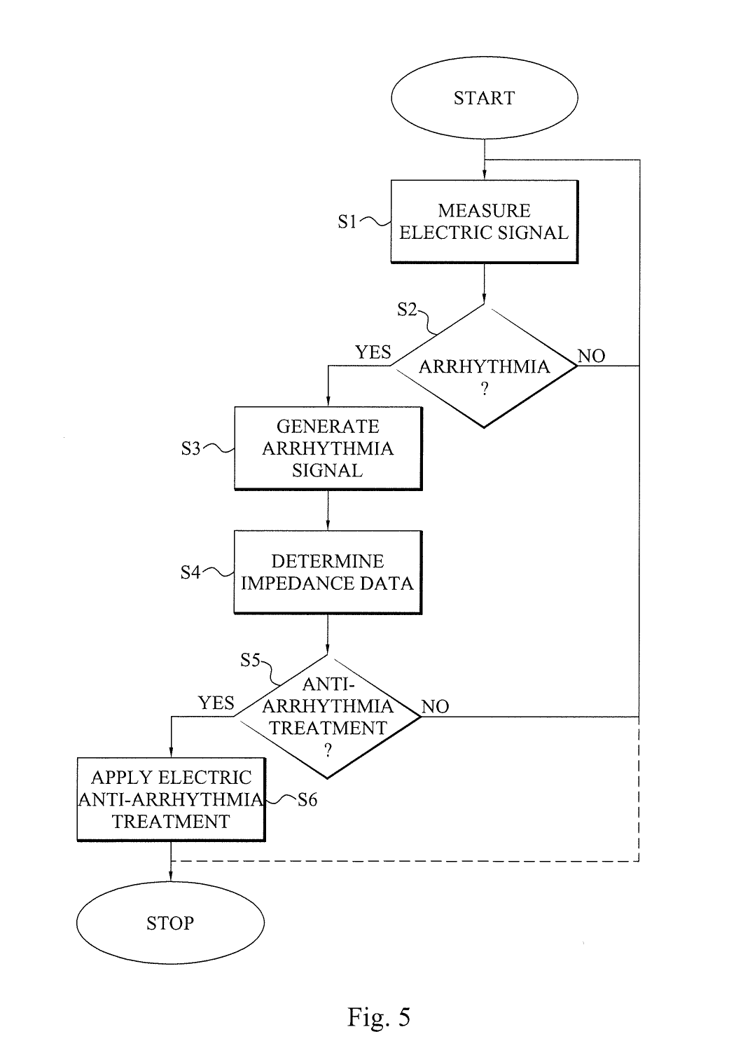

FIG. 5 is a flow diagram illustrating a method of treating arrhythmia of a heart in a subject according to the present invention. The method starts in step S1, where electric signals, preferably intrinsic electric signals, from the heart are measured. The measured electric signals are employed in a next step S2 for detecting the presence of an arrhythmia state of the heart. If the signals do not indicate any arrhythmia the method returns to step S1. The measurements of step can therefore be performed periodically or intermittently, such as at predetermined time intervals.

If an arrhythmia state of the heart is detected in step S2 based on the measured electric signal the method continues to step S3. Step S3 involves generating an arrhythmia signal based on the detected arrhythmia state. In other words, this signal is representative of the presence of an arrhythmia state in the patient. A next step S4 determines impedance data representative of an erythrocyte aggregation level in blood. This erythrocyte aggregation level is determined for blood present in a cavity of the patient, preferably a cavity connected to the cardiovascular system of the patient, such as in at least one heart chamber.

The impedance data determination of step S4 can be performed independent on the arrhythmia detection of step S2. In such a case, the impedance data can be determined periodically or intermittently, such as at predetermined time intervals, in similarity to the signal measurements of step S1. However, in order to save battery power required for generating the impedance data, the determination of step S4 can be performed based on the arrhythmia signal generated in step S3. In such a case, impedance data is then only determined in connection with a detected arrhythmia event.

A next step S5 involves a conditional anti-arrhythmia treatment that is conditioned based on the determined impedance data and the generated arrhythmia signal. In a typical embodiment, this conditional treatment involves conditionally applying electric anti-arrhythmia treatment signal(s) to at least a portion of the patient's heart based on the aggregation status (impedance data) and arrhythmia state (arrhythmia signal). Step S5 can, thus, involve selecting whether to directly apply the electric anti-arrhythmia treatment, such as defibrillation, cardioversion or anti-arrhythmia pacing, or postpone the treatment or apply an alternative treatment that reduces the risk of obstructing blood vessels by blood clots that otherwise can occur following the traditional anti-arrhythmia treatment. Thus, if the erythrocyte aggregation level is within a normal, non-risk range as determined based on the impedance data, the method continues from step S5 to step S6, where the electric anti-arrhythmia treatment is applied. However, if the impedance data indicates a high aggregation level with an associated significant risk of causing blood vessel obstructions by blood clots and aggregates, the method could continue from step S5 back to step S1 to thereby postpone or at least temporarily put any anti-arrhythmia treatment on hold. Alternatively, an alternative treatment could be applied following step S5, which is more adapted to the current aggregation status.

In the former case, the planned anti-arrhythmia treatment can be postponed for a defined time period, for instance by, counting down a clock. The anti-arrhythmia treatment could thereafter be applied once the defined time period has elapsed. A more preferred embodiment, however, returns the method to step S1 to anew test the presence of any arrhythmia. The advantage of such approach is that the arrhythmia could spontaneously revert back to healthy normal condition without the need for any anti-arrhythmia treatment during the postponed time period. If the arrhythmia, though, is present, the method continues from step S2 through steps S3-S5. If the erythrocyte aggregation level is still high, a new postponing period may be triggered. This procedure can be continued until either the arrhythmia spontaneously reverts, becomes potentially fatal or the aggregation level reduces (in which case anti-arrhythmia treatment can safely be applied in step S6). In an alternative approach, the treatment postponing is allowed no more than a defined maximum number of times before the electric anti-arrhythmia treatment is applied in step S6 even though the impedance data indicates a higher aggregation level than normal.

FIG. 6 is a flow diagram illustrating an additional step of the treatment method of FIG. 5. The method continues from step S1 of FIG. 5. The next step S10 estimates a heart rate based on the electric signals measured or sensed from the heart. In a preferred embodiment, the heart rate is estimated in step S10 from an electrocardiogram signal measured from the heart. The method then continues to step S2 of FIG. 5, where the arrhythmia detection is performed based on a comparison of the estimated heart rate with a threshold rate value or range.

FIG. 7 is a flow diagram illustrating the impedance determining step of FIG. 5 in more detail. The method continues from step S3 of FIG. 5. In a next step S20, a current or voltage (pulse) signal is applied over two electrodes, preferably based on the detection of the arrhythmia. A resulting voltage or current signal is then measured using two electrodes in step S21. The applied signal and the measured resulting signal are then processed in step S22 for generating impedance data representative of the erythrocyte aggregation level of blood present in the measurement cavity. The processing preferably involves determining the impedance data as average data over multiple time periods of different heart beats. The method then continues to step S5 of FIG. 7.

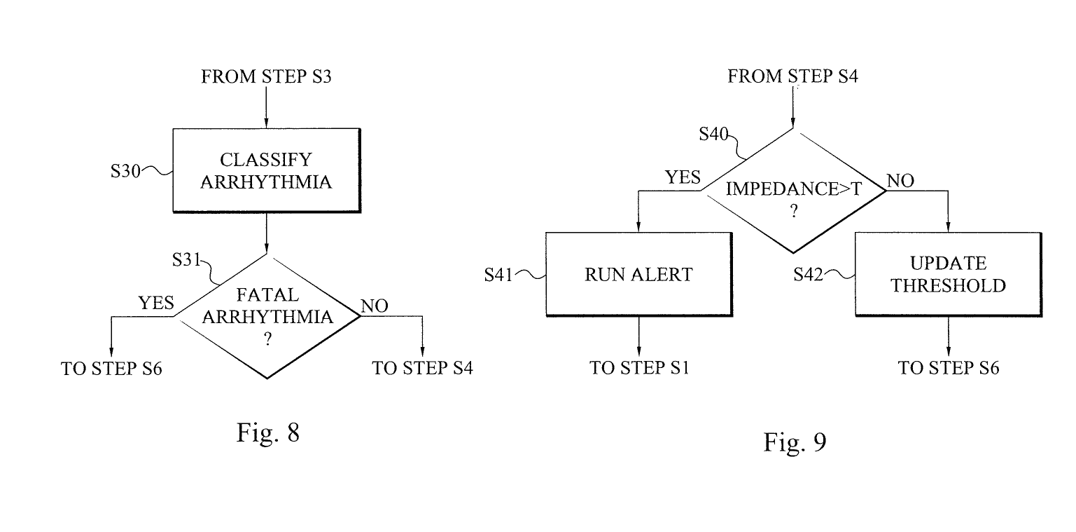

FIG. 8 is a flow diagram illustrating additional steps of the treating method of FIG. 5. The method continues from step S3 in FIG. 5. A next step S30 classifies the detected arrhythmia and generates classification data representative of the estimated severity of the arrhythmia. In a preferred embodiment, the classification is between potentially fatal and potentially non-fatal arrhythmia. Another embodiment utilizes a more detailed arrhythmia classification by for instance also discriminating between atrial and ventricular arrhythmias, tachy- and bradyarrhythmias and/or fibrillation and non-fibrillation types of arrhythmia. In such a case, the conditional anti-arrhythmia treatment of the present invention is conditionally applied based on the arrhythmia signal, the impedance data and the classification data.

The method therefore continues to step S31, where discrimination between fatal and non-fatal arrhythmias is performed based on the classification data. If the arrhythmia is regarded as potentially fatal in step S31, such as ventricular fibrillation, the method continues to step S6, where electric anti-arrhythmia treatment is applied to the heart regardless of the current erythrocyte aggregation status.

However, if a non-fatal arrhythmia is detected and classified, the method continues from step S31 to step S4 of FIG. 5, where impedance data is determined and the anti-arrhythmia treatment is conditioned based on the impedance data and the arrhythmia signal.

FIG. 9 illustrates an embodiment of the conditional anti-arrhythmia treatment step of FIG. 5 in more detail. The method continues from step S4 in FIG. 5. A next step S40 compares the determined impedance data with an impedance threshold or template. If there is a significant difference between the determined data and the threshold/template, which indicates high blood aggregation risk, the method continues to step S41. Step S41 involves providing an alert signal that informs the patient or a physician of the presence of the arrhythmia and the high aggregation level. The alert signal can, as previously mentioned, be an electric signal sent to an external unit, an audio signal and/or tactile signal. The patient can therefore initiate anti-thrombotic actions during the treatment postponement to thereby try to reduce the aggregation status of the blood before any anti-arrhythmia treatment is initiated.

If there is no significant difference between impedance data and the threshold/template in step S40, anti-arrhythmia treatment can be initiated in step S6 of FIG. 5. Optionally, the threshold/template can be updated in step S42 based on the determined impedance data.

The treatment method may also involve usage of different thresholds/templates adapted for different patient statuses, such as posture and activity level, as previously mentioned.

Although modifications and changes may be suggested by those skilled in the art, it is the intention of the inventors to embody within the patent warranted heron all changes and modifications as reasonably and properly come within the scope of their contribution to the art.

We claim as our invention:

1. An implantable medical device comprising:
an arrhythmia detector that detects an arrhythmia state of a heart of a subject based on an electric signal measured from said heart and for generating an arrhythmia signal based on said detected arrhythmia state;
an impedance determining unit for determining impedance data representative of an erythrocyte aggregation level of blood present in a cavity in said subject; and
an anti-arrhythmia unit that applies electric anti-arrhythmia treatment to at least a portion of said heart and being conditionally operable based on said arrhythmia signal and said impedance data.

2. The device according to claim 1, wherein said impedance determining unit is responsive to said arrhythmia signal and is arranged for determining said impedance data based on said arrhythmia signal.

3. The device according to claim 1, wherein said impedance determining unit determines said impedance data representative of said erythrocyte aggregation level of blood present in a chamber of said heart.

4. The device according to claim 1, further comprising an arrhythmia classifier that generates, based on said arrhythmia signal, classification data representative of an estimated severity of said arrhythmia state, wherein said anti-arrhythmia unit is conditionally operable based on said arrhythmia signal, said impedance data and said classification data.

5. The device according to claim 4, wherein said anti-arrhythmia unit is operable for applying said electric anti-arrhythmia treatment to said at least a portion of said heart if said classification data indicates a potentially fatal arrhythmia state.

6. The device according to claim 4, wherein said anti-arrhythmia unit is operable for conditionally applying said electric anti-arrhythmia treatment to said at least a portion of said heart based on said arrhythmia signal and said impedance data if said classification data indicates a potentially non-fatal arrhythmia state.

7. The device according to claim 1, wherein said anti-arrhythmia unit postpones, based on said impedance data, an application of said electric anti-arrhythmia treatment to said at least a portion of said heart for a defined time period.

8. The device according to claim 7, further comprising an aggregation detector that compares said impedance data with an impedance threshold and said anti-arrhythmia unit postpones said application of said electric anti-arrhythmia treatment to said at least a portion of said heart for said defined time period if said impedance data exceeds said impedance threshold.

9. The device according to claim 8, further comprising a threshold processor that generates said impedance threshold as an average of multiple previous impedance data determined by said impedance determining unit at multiple previous time instances.

10. The device according to claim 7, further comprising an alert unit that provides an alert signal if said anti-arrhythmia unit postpones said application of said electric anti-arrhythmia treatment to said at least a portion of said heart.

11. A method for treating an arrhythmia state of a heart of a subject, said method comprising:
measuring an electric signal from said heart;
detecting an arrhythmia state of said heart based on said electric signal;
generating an arrhythmia signal based on said detected arrhythmia state;
determining impedance data representative of an erythrocyte aggregation level of blood present in a cavity in said subject; and
conditionally applying electric anti-arrhythmia treatment to at least a portion of said heart based on said arrhythmia signal and said impedance data.

12. The method according to claim 11, further comprising estimating a heart rate of said heart based on an electrocardiogram signal measured for said heart, wherein said detecting step comprises detecting said arrhythmia state based on a comparison of said estimated heart rate and a threshold rate.

13. The method according to claim 11, wherein said determining step is performed based on said arrhythmia signal.

14. The method according to claim 11, further comprising generating, based on said arrhythmia signal, classification data representative of an estimated severity of said arrhythmia state, wherein said conditionally applying step comprises conditionally applying said electric anti-arrhythmia treatment to at least said portion of said heart based on said arrhythmia signal, said impedance data and said classification data.

15. The method according to claim 14, wherein said conditionally applying step comprises applying said electric anti-arrhythmia treatment to said at least a portion of said heart if said classification data indicates a potentially fatal arrhythmia state.

16. The method according to claim 14, wherein said conditionally applying step comprises conditionally applying said electric anti-arrhythmia treatment to said at least a portion of said heart based on said arrhythmia signal and said impedance data if said classification data indicates a potentially non-fatal arrhythmia state.

17. The method according to claim 11, wherein said conditionally applying step comprises postponing, based on said impedance data, an application of said electric anti-arrhythmia treatment to said at least a portion of said heart for a defined time period.

18. The method according to claim 17, further comprising comparing said impedance data with an impedance threshold, wherein said postponing step comprises postponing said application of said electric anti-arrhythmia treatment to said at least a portion of said heart for said defined time period if said impedance data exceeds said impedance threshold.

19. The method according to claim 18, further comprising generating said impedance threshold as an average of multiple previous impedance data determined at multiple previous time instances.

20. The method according to claim 17, further comprising providing an alert signal if said application of said electric anti-arrhythmia treatment to said at least a portion of said heart is postponed.
